(19) United States
(12) Patent Application Publication
(10) Pub. No.: US 2013/0123715 A1
(43) Pub. Date: May 16, 2013

(54) Indwelling catheter probe incorporating proanthocyanidines

(71) Applicants: Gunter HAESAERTS, Rueil Malmaison (FR); Henri BOTTO, Paris (FR)

(72) Inventors: Gunter HAESAERTS, Rueil Malmaison (FR); Henri BOTTO, Paris (FR)

(21) Appl. No.: 13/681,990

(22) Filed: Nov. 20, 2012

(30) Foreign Application Priority Data
May 12, 2010 (FR) 10.02028

(51) Int. Cl. A61M 25/00

(52) U.S. Cl. CPC A61M 25/0017
USPC 604/265; 604/544; 427/2.3

(57) ABSTRACT

An indwelling catheter probe, wherein it incorporates, at its external and/or internal surface, an extract of Vaccinium macrocarpon containing proanthocyanidines applied either by adsorption, by absorption, by coating or by any other application process, namely any physico-chemical process, said catheter probe thus treated ensuring the prevention or inhibition of the formation of a bacterial biofilm at its surface.

### 1. FIELD OF THE INVENTION

The technical scope of the present invention is that of probes and catheters and more specifically that of indwelling urinary and vascular catheters.

### 2. DESCRIPTION OF THE RELATED ART

The urinary tract is the principal site of nosocomial urinary tract infections (NUTIs), that is to say, those related to diagnostic and/or therapeutic care and these NUTIs represent 40% of all health-care related infections and thus have become a genuine scourge to Public Health.

The incidence of these NUTIs is namely favored by the presence of a urinary catheter and the risk of infection increases with the length of time the catheter is in place.

These NUTIs are generally asymptomatic, however they may be symptomatic in which case they are accompanied by one or several infections in the urinary tract such as a bladder infection or cystitis, a kidney infection or pyelonephritis, a prostate infection or prostatitis, an infection of the epididymis or epididymititis, an infection of the testicle or orchitis and/or a septicaemia of urinary origin called urosepsis.

Additionally, these NUTIs constitute a reservoir of germs that risk being transmitted to healthy persons.

Lastly, these NUTIs contraindicate cystoscopic diagnosis and/or therapeutic care because of the major risk of serious complications.

The bacteria Escherichia coli (E. coli) is the dominant bacteria in cases of NUTIs, even if its frequency is less than that observed in community-acquired urinary tract infections. Where the presence of E. coli has dropped that of other germs such as Enterococcus spp, Staphylococcus spp or other gram-negative bacilli has increased.

The germs at the origin of these NUTIs have a higher rate of antibiotic-resistant bacterial strains than that observed for community-acquired urinary tract infections.

Thus, these NUTIs generate substantial extra direct and indirect treatment costs.

In the present application, the applicant has concentrated more particularly on indwelling catheters intended to be positioned in the patient's urinary tract on a short or long term basis. The applicant also considers catheters intended to be positioned in a patient's veins or arteries.

In the present applicant, the terms “probe” or “catheter” are used indifferently to describe the device according to the invention.

The use of catheters coated with one or several active substances has been proposed in the aim of preventing the formation of a biofilm.

Thus, the article by P. Tenka et al. (EAU Update Series 2 (2004) 106-115) describes the fundamentals of the prevention and treatment of nosocomial urinary tract infections and the cases where urinary catheters are used. To avoid the formation of the biofilm, the technique consists either in modifying the surface of the catheter by incorporating biocides or antibiotics into the material of said catheter to ensure the controlled diffusion of the substances, or in developing materials that have surface properties that prevent bacteria from adhering to them. The substances which can be used to coat catheters are antibiotics for catheters for short-term use, silver oxide or silver alloy which inhibits the bacteriuria, phosphorylcholine which effectively inhibits the formation of the biofilm or heparin for catheters intended for long-term use.

The study performed by U.S. Ha, Y-H Cho (International Journal of Antimicrobial Agents 28(2006) 485-490) reviews the strategies used to prevent catheter-related urinary tract infection and in particular catheters coated with different materials targeting the pathogenicity of the biofilms. The catheter may here also be coated in silver but the use of this type of catheter is not supported by quality data and the issue of resistance to silver may arise. The catheter may be coated with hydrogels, which are macromolecular polymers able to absorb relatively large volumes of liquid inside their polymeric structures causing the formation of a thin aqueous film and improving the smooth aspect and the surface lubrication of the catheter probe thereby reducing the adhesion of the bacteria. However, there is insufficient proof either to support or recommend the use of this type of catheter. Lastly, the catheter may be coated with antibiotics, for example gentamicin, norfloxacin or nitrofurazone, which is effective for catheters intended for short-term use in order to prevent or inhibit the outbreak of catheter-related urinary tract infections by acting on the biofilms. However, the use of antibiotics poses the problems of the appearance of resistant strains, a phenomenon which has already spread to numerous bacterial strains. The article by S. Tamilvanan et al. (Journal of Controlled Release 128(2008) 2-22) describes the prevention means adopted to eradicate biofilms in nosocomial infections related to medical implants. Here again the strategy consists in modifying the catheter's surface by impregnation or coating with various substances such as antibacterial agents, antiseptics and/or metals. These active substances may be incorporated into polymer-based vectors or lipid-based vectors (called liposomes) to be delivered at sufficiently therapeutic doses to the biofilm. Antimicrobial coatings are intended to prevent the formation of the biofilm by eliminating early colonizing bacteria. Liposomes are essentially studied to enhance the anti-adhesive properties of the implant materials and to target the antimicrobial agents onto the biofilms in cases of intercellular infections. Polymeric vectors are developed to eradicate infections associated with biofilm specifically in implants and in the periodontal cavity by the local delivery of the enclosed antibiotic substances. However, as previously mentioned, the use of antimicrobial substances suffers the drawback of increasing the risk of bacterial resistance and the potential of the vectors described in the eradication of the biofilm is not fully established and requires additional trials as well as the authorization of the public health authorities before the technique can be administered to human patients.

Furthermore, supports coated with proanthocyanidines (PACs) derived from cranberry (Vaccinium macrocarpon) to inhibit the adhesion of bacteria are described.

Thus, the study performed by Eydelnant and Tufenkji (Langmuir 24 (2008) 10273-10281) aims to determine if the adhesion of bacteria to common biomaterials can be reduced by a treatment based on PACs derived from cranberry (V. macrocarpon). The bacteria under study are strains of E. coli and the biomaterials studied are polyvinylchloride (PVC) and Polytetrafluoroethylene (PFTE). Experiments consist in treating either the bacteria, or the biomaterials, or both bacteria and biomaterials using PACs placed in solution in a tampon, the control being the absence of treatment of the bacteria and the biomaterial. Cellular adhesion is thereafter evaluated for each treatment. This study demonstrates that the PACs are effectively adsorbed on the biomaterials and that they reduce the adhesion of the bacteria to said biomaterials, but that this mechanism is partly non-specific. Nevertheless, the extract of PACs used here is of a mean molecular weight of 15 kDa and corresponds to a quasi-pure extract of PACs prepared in a laboratory in conditions that can not be transposed to an industrial setting, namely because of the use of acetone. Additionally, the results obtained only show the effectiveness of PACs in the anti-adhesive of the bacteria and the conclusion clearly explains that additional research is being pursued to explore the use of these PACs in the prevention of the biofilm formation.

Lastly, French patent FR-2874826 of the applicant is known and concerns the production of a preparation in gel form intended to coat or impregnate probes so as to enable the diffusion of the V. macrocarpon vegetal extract which constitutes the active substance and which presents anti-adhesive properties with respect to pathogenic microbes or those from nosocomial infections. In addition to the extract of V. macrocarpon at 1% PACs, the gel may contain other active substances with a similar or synergetic action and also excipients. This gel is arranged in a casing enclosing the catheter probe to ensure the constant impregnation of said catheter probe by contact with the gel, the extract being able to coat or insert itself into the microporous surface of the catheter probe, without the efficiency of this gel being able to be determined with any certainty. It is indicated, however, that the extract of V. macrocarpon is spread in the form of a liposome upon the surface of the catheter probe and here is used as a destructive agent for the bacterial adhesins thereby inhibiting their fixing onto the inner lining of the organism. Additionally, in this patent, no proof is produced as to the effective impregnation of the V. macrocarpon nor to the spreading of this extract on the catheter probe's surface in the form of liposomes. Lastly, this patent produces no proof of the effectiveness of the method in preventing the formation of a biofilm.

As can be observed, the state of the art shows that the formation of a biofilm poses a veritable problem in human medicine, such problem remaining unresolved or only partially resolved. The different attempts mentioned above show the diversity of the fields of research explored. Moreover, there have been no studies of the practical application to the treatment for patients of urinary tract infections associated with the presence of a catheter and the formation of a biofilm.

### SUMMARY OF THE INVENTION

In the present invention, the applicant surprisingly focuses not on the anti-adhesive action of the extract with 1% PACs against bacteria, but on the inhibiting action with regard to the formation of the biofilm on the surface of a catheter probe of an extract of V. macrocarpon with a higher level of PACs.

The present invention thus aims to overcome the drawbacks to prior art by supplying a catheter probe intended to inhibit or prevent the formation of a bacterial biofilm on its surface.

The invention thus relates to an indwelling catheter probe, wherein it incorporates, at its external and/or internal surface, an extract of V. macrocarpon containing proanthocyanidines applied either by adsorption, by absorption, by coating or by any other application process, namely any physico-chemical process, said catheter probe thus treated ensuring the prevention or inhibition of the formation of a bacterial biofilm at its surface.

According to one characteristic of the invention, the extract of Vaccinium macrocarpon contains approximately between 16 and 30% in mass of proanthocyanidines.

According to another characteristic of the invention, the extract of Vaccinium macrocarpon comprises 18% in mass of proanthocyanidines.

According to another characteristic of the invention, the extract of Vaccinium macrocarpon comprises 20% in mass of proanthocyanidines.

According to another characteristic of the invention, the extract of Vaccinium macrocarpon comprises 26% in mass of proanthocyanidines.

According to another characteristic of the invention, the extract of Vaccinium macrocarpon comprises 30% in mass of proanthocyanidines.

The invention also relates to a preparation process for a catheter probe, wherein:
the extract of Vaccinium macrocarpon is mixed in powder form with a suitable solvent such as water or other inert solvent,
the probe and solution prepared using the Vaccinium macrocarpon extract are brought into mutual contact for at least 24 h at a temperature of 37° C. so as to enable the adsorption or other means (for example absorption or coating) of fixing the Vaccinium macrocarpon extract and the proanthocyanidines to the surface of said probe,
the catheter probe thus treated in lastly inserted into position in the patient.

The invention also relates to the application of the catheter probe to the reduction and/or elimination of urinary tract infections caused by the presence of indwelling catheters.

The invention also relates to the application of the catheter probe to the reduction and/or elimination of vascular infections caused by the presence of indwelling vascular catheters.

A first advantage of the invention lies in the inhibition of the formation of the bacterial biofilm at the probe's surface which constitutes over time a source of infection.

Another advantage of the invention lies in the retardation of the appearance of the bacterial biofilm at the surface of the probe.

Another advantage of the invention lies in the reduction of the nosocomial infection rate thanks to the reduction in the bacterial biofilm.

Yet another advantage of the invention is the provision of simple, effective and perfectly innocuous means to fight against bacterial biofilms that suffer no side effects for the patient with an indwelling catheter and that do not increase the resistance of bacteria to antibiotics.

Other characteristics, particulars and advantages of the invention will become more apparent from the description given hereafter by way of illustration of the specific embodiments of the invention.

Two types of bacterial population can be observed in the urinary tract. Firstly, so-called “planktonic” bacteria, that is to say in suspension in the urine medium, which are metabolically active and which remain more often than not sensitive to the action of antibiotics. Secondly, quiescent bacteria are deeply embedded in a biofilm and are generally insensitive to treatment and namely to antibiotics. It is this second category of bacteria which is responsible for the urinary infections suffered by patients with indwelling catheters. In fact, the bacteria embedded in the biofilm develops away from antimicrobial agents and are able to survive a concentration of antimicrobial agents 1000 to 1500 times greater than that required to kill planktonic bacteria.

Whether the urinary infection is acquired endoluminally or extraluminally, the bacteria colonising the urinary probe increase in the form of micro-colonies embedded in a bacterial biofilm that protects them. By bacterial biofilm we refer to a build-up of microorganisms and their extracellular product that form a structured community on the solid surface of the probe or catheter. The formation of this bacterial biofilm on a solid support follows a well-established sequence. Firstly, when a catheter probe is inserted into the urethra, a packaging film composed of water, electrolytes and organic substances naturally forms on its surface. Uropathogenic bacteria may be introduced into the urinary tract along with the probe. These bacteria thus adhere to the surface of the urinary catheter, which constitutes a foreign body, and become anchored there. These adhesive bacteria multiply and secrete an extracellular polysaccharide matrix called “slime” or “glycocalyx” that is in the form of a viscous deposit. The bacterial biofilm thus formed in turn favors the adhesion of bacteria to the probe's surface and constitutes a sanctuary protecting these bacteria from the patient's defense mechanisms and from antimicrobial agents. Additionally, the patient's urinary salts and proteins, such as Tamm-Horsfall protein, are incorporated into this matrix forming encrustations on the catheter probe's surface, encrustations which are calcium deposits that can form over a period as short as eight days and which solidify at the surface of the probe thereby further structuring the biofilm. Patients with indwelling urinary catheters are observed to suffer a rate of urinary tract infection due to the introduction of these external germs in 15 to 20% of cases.

The aim of the invention is precisely to inhibit and/or delay the formation of this bacterial biofilm and to reduce its development.

The V. macrocarpon berry contains many phenolic substances of which around 40% are condensed tannins called proanthocyanidines (PACs). These PACs are oligomers (dimers to decamers) and (epi)catechine polymers and can be type A or type B. Type A PACs, present only in the berries of this plant, are characterized by the presence of a double inter-flavin link and are the only ones to possess bacterial anti-adhesive properties.

The PACs extract used in the invention is an extract that comprises all the phenols obtained from concentrated cranberry juice (V. macrocarpon). This extract contains all the PACs from the juice, which represents around 16 to 30% of the total mass of this extract of V. macrocarpon measured using the BL-DMAC method (Dimethylaminocinnamaldehyde Color Reaction developed by Brunswick Laboratories) (www.dmac-asso.org). The extraction and purification process for this extract using distilled water and ethanol ensures optimal preservation of all the polyphenols. The extract obtained is in the form of a very fine powder with a particle size where more than 90% passes through a 100 μm mesh sieve.

Contrary to the extract used by EYDELNANT, which is impossible to prepare on an industrial scale and is unavailable on the market, the PACs extract according to the invention is perfectly adapted to industrial production, safeguards public health and is, moreover, authorized by the public health authorities.

To prepare the catheter probe according to the invention, the extract of V. macrocarpon in powder form is mixed with solvent, water for example. The probe is impregnated for at least 24 hours at a temperature of 37° C. so as to enable the V. macrocarpon extract to be adsorbed or absorbed on the surface of the probe.

The V. macrocarpon extract-based solution containing the PACs to be integrated to the probe's surface additionally encloses suitable excipients, auxiliary agents and/or additives in addition to the active substance.

The PACs may be integrated into the probe either by adsorption, by absorption, coating or any other application, namely physico-chemical, process. These PACs are present on the probe's surface in continuous and/or discontinuous layers.

The catheter probe according to the invention may be constituted by classical material such as silicones, polyurethanes, polyvinylchloride (PVC), Polytetrafluoroethylene (PTFE), polypropylene and/or any other material used in this domain.

### BRIEF DESCRIPTION OF THE DRAWING

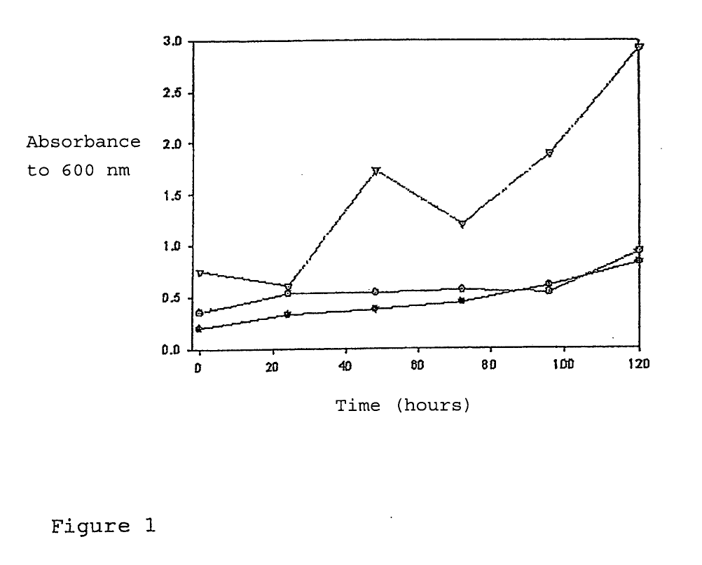

The invention will become more apparent from the following additional description of practical embodiments where FIG. 1 illustrates the absorbance results obtained as a function of time.

### DETAILED DESCRIPTION OF PREFERRED EMBODIMENTS

As explained previously, the applicant has observed that a catheter probe according to the invention prevents the formation of the bacterial biofilm at its external and/or internal surface and consequently limits the occurrence of urinary tract infections in patients with indwelling catheters.

The examples given hereafter clearly highlight the great advantages of the invention and the progress it brings to human or animal medical health. Trials have been performed on strains of E. coli and namely on E. coli B037, which is a virulent clinical strain highly resistant to antibiotics, as well as on strains of Staphylococcus, namely S. aureus.

In the following examples, the bacterial solution is composed by bacteria having been cultivated in a culture medium, such as Luria Bertani (lysogeny) broth, with no V. macrocarpon extract, and extracted in the middle or at the end of the exponential growth phase.

### EXAMPLE 1

The objective here is to confirm that the presence of the V. macrocarpon extract on the probe's surface effectively inhibits or prevents the formation of a bacterial biofilm.

The polypropylene probes are disinfected using 70% ethanol and are exposed to ultraviolet rays for 30 minutes so as to eliminate any microorganisms that may be present on the probe.

The probes are put into contact with a V. macrocarpon extract-based solution as described previously that contains approximately 18% of PACs and whose concentration is of 0.12 mg/ml. The probes are left in contact with the solution and are incubated at 37° C. for 24 hours thereby enabling the V. macrocarpon extract, and more specifically the PACs contained it in, to be adsorbed (or absorbed) on the external and internal surface of the probes. After incubation, the excess V. macrocarpon extract is removed and the probes are dried. The controls are constituted here by disinfected probes that are not treated with the V. macrocarpon extract.

3 ml of an E. coli B37 bacterial solution is deposited on the probes and these are left to incubate for 0, 24, 48, 72, 96 and 120 hours. The bacterial cells are colored with a 0.3% crystal violet solution for 15 minutes and the planktonic bacteria are eliminated by being washed three times in ultrapure water. The bacteria forming the biofilm are detached from the probe by being immersed in an ethanol/acetone solution for 15 minutes. The absorbance is then measured at a wavelength of 600 nm.

FIG. 1 shows the absorbance measured at 600 nm as a function of time for this treatment. The symbols express the following conditions:
== With treatment
-▾- Without treatment

In the case of untreated probes, the absorbance values increase over time to reach the value of 3 after 120 hours. On the contrary, for treated probes, the absorbance values are observed to not exceed 1 even after 120 hours of exposure to the bacteria. Knowing that the presence of a biofilm is characterized by an absorbance of at least 1 for this wavelength (O'Toole method “G. A. O'Toole, R. Kolter, Initiation of biofilm formation in Pseudomonas fluorescens WCS365 proceeds via multiple, convergent signaling pathways: a genetic analysis, Molecular Microbiology, 28 (1998) 449-461), we can conclude that the treatment of the probes according to the invention enables the formation of the biofilm at their surfaces to be inhibited for at least 5 to 6 consecutive days.

Equivalent results are obtained for Staphylococcus aureus.

### EXAMPLE 2

The objective here is to determine if the V. macrocarpon extract has been effectively adsorbed (or absorbed) by the probe or if this extract can be easily eliminated after treatment. The probes are treated as explained in Example 1. However, after incubation in the V. macrocarpon extract-based solution, the probes are washed using ultrapure water and then dried.

3 ml of bacterial solution containing E. coli B37 is deposited on the probes and left to incubate for 0, 24, 48, 72, 96 and 120 hours. After incubation, the probes are colored and treated as indicated in Example 1, then the absorbance is measured at 600 nm.

FIG. 1 also shows the absorbance measured at 600 nm over time for this treatment. The symbols express the following conditions:
-- With treatment followed by washing

As explained previously, in the case of untreated probes, the absorbance values increase over time to reach the value of 3 after 120 hours. For the probes which are treated and then washed, however, the absorbance values do not exceed the value of 1 even after 120 hours of exposure to the bacteria. Washing the probes using ultrapure water before contact with the bacterial solution does not affect the absorbance values, thereby demonstrating that the V. macrocarpon extract is not eliminated by washing. From this, the non-specific adsorption (or absorption) can be deduced to be high and to last for 5 to 6 consecutive days.

### EXAMPLE 3

An extract of V. macrocarpon is used with 20% PACs and the probe is treated in the same manner. The same absorbance measurements are made and no value exceeds 1. The formation of a biofilm is thus inhibited.

### EXAMPLE 4

An extract of V. macrocarpon is used with 26% PACs and the probe is treated in the same manner. The same absorbance measurements are made and no value exceeds 1. The formation of a biofilm is thus inhibited.

### EXAMPLE 5

An extract of V. macrocarpon is used with 30% PACs and the probe is treated in the same manner. The same absorbance measurements are made and no value exceeds 1. The formation of a biofilm is thus inhibited.

The catheter probes according to the invention thus enable the formation of the bacterial biofilm to be inhibited even with respect to antibiotic-resistant strains of E. coli as well as to S. aureus. The V. macrocarpon extract is effectively adsorbed (or absorbed) by the probe for at least 5 to 6 days, thereby confirming it usefulness for indwelling catheters.

The invention thus procures a significant improvement to the conditions of treatment for patients with indwelling catheters, without worsening the phenomenon of resistance. The invention thus constitutes effective and reliable means to prevent nosocomial urinary tract infections and their resulting complications.

The invention such as described above can also be applied to vascular catheters to overcome local or general problems such as septicaemia or risks of secondary seats of infection caused by the presence of indwelling vascular catheters.

1. An indwelling catheter probe, wherein it incorporates, at its external and/or internal surface, an extract of Vaccinium macrocarpon containing proanthocyanidines applied either by adsorption, by absorption, by coating or by any other application process, namely any physico-chemical process, said catheter probe thus treated ensuring the prevention or inhibition of the formation of a bacterial biofilm at its surface.

2. A catheter probe according to claim 1, wherein the extract of Vaccinium macrocarpon contains approximately between 16 and 30% in mass of proanthocyanidines.

3. A catheter probe according to claim 2, wherein the extract of Vaccinium macrocarpon comprises 18% in mass of proanthocyanidines.

4. A catheter probe according to claim 2, wherein the extract of Vaccinium macrocarpon comprises 20% in mass of proanthocyanidines.

5. A catheter probe according to claim 2, wherein the extract of Vaccinium macrocarpon comprises 26% in mass of proanthocyanidines.

6. A catheter probe according to claim 2, wherein the extract of Vaccinium macrocarpon comprises 30% in mass of proanthocyanidines.

7. A preparation process for a catheter probe according to claim 1, wherein:
the extract of Vaccinium macrocarpon is mixed in powder form with a suitable solvent such as water or other inert solvent,
the probe and solution prepared using the Vaccinium macrocarpon extract are brought into mutual contact for at least 24 h at a temperature of 37° C. so as to enable the adsorption or other means (for example absorption or coating)of fixing the Vaccinium macrocarpon extract and the proanthocyanidines to the surface of said probe,
the catheter probe thus treated in lastly inserted into position in the patient.

8. The application of the catheter probe according to claim 1 to the reduction and/or elimination of urinary tract infections caused by the presence of indwelling catheters.

9. The application of the catheter probe according to claim 1 to the reduction and/or elimination of vascular infections caused by the presence of indwelling vascular catheters.
